(19) United States
(12) Patent Application Publication
(10) Pub. No.: US 2011/0124912 A1
(43) Pub. Date: May 26, 2011

(54) MONOETHERIFIED DIOLS OF DIAMONDOIDS

(75) Inventors: Peter R. Schreiner, Wettenberg (DE); Hartmut Schwertfeger, Asslar (DE)

(73) Assignee: JUSTUS-LIEBIG-UNIVERSITAT GIESSEN, Giessen (DE)

(21) Appl. No.: 12/996,399

(22) PCT Filed: Jun. 2, 2009
(86) PCT No.: PCT/EP09/56746

§ 371 Date: Jan. 31, 2011

(30) Foreign Application Priority Data
Jun. 7, 2008 (DE) 10 2008 027 341.4

(51) Int. Cl. C07C 227/02; C07C 43/196; C07C 41/09; C07C 231/06; C07C 213/02

(52) U.S. Cl. 562/498; 568/665; 564/210; 564/458

(57) ABSTRACT

The invention at hand describes functionalized diols of diamondoids, in which one of the two hydroxy groups is masked by a protective group, as well as methods for producing these functionalized diols. The protective group is a group —CHR¹R², wherein R¹ and R² stand for alkyl groups and the protective group comprises at least one halogen atom. The monoethers of the diamondoid diols according to the present invention are produced by reacting the diamondoid diol with a halogenated alcohol CHOHR¹R² in the presence of a catalyst acid.

The monoetherified diols allow for the targeted production of derivatives of diamondoids, for example, of the corresponding aminoalcohols and aminocarboxylic acids. For that purpose, the diamondoid monoether is reacted in a first step with a halogen nitrile in a Ritter reaction to the corresponding monoether amide. From this monoether amide, the corresponding aminoalcohol can be produced by reacting the protective group —CHR¹R² first with trifluoroacetic acid to the alkanoyloxy group and by subsequently obtaining the aminoalcohol by reaction with thiourea, ethanol and glacial acetic acid.

The aminoalcohol can be reacted with a sulfuric acid/formic acid or oleum/formic acid to the corresponding aminocarboxylic acid.

The amino, hydroxy and carboxylic groups of the diamondoids can be converted into many other functional groups.

The invention at hand describes functionalized diols of diamondoids, in which one of the two hydroxy groups is masked by a protective group, as well as methods for producing these functionalized diols. The protective group is a —CHR¹R² group, wherein R¹ stands for an alkyl group and R² for hydrogen or an alkyl group. The protective group contains at least one halogen atom, preferably fluorine. The monoetherified diols allow for the targeted production of derivatives of diamondoids, for example of the corresponding aminoalcohols and aminocarboxylic acids.

### DESCRIPTION OF AND INTRODUCTION TO THE GENERAL FIELD OF THE INVENTION

The invention at hand relates to functionalized diamondoids. These are monoetherified diols of diamondoids which are suitable for comprising, optionally, further covalently bound functional groups in addition to the ether and hydroxyl group.

### STATE OF THE ART

Diamondoids are cage-like substituted and unsubstituted compounds of the adamantane series. The adamantane series comprises adamantane, diamantane, triamantane, tetramantane, pentamantane, hexamantane, heptamantane, octamantane, nonamantane, decamantane, undecamantane and similar compounds as well as all isomers and stereoisomers.

These compounds comprise a diamondoid topology, i.e. the arrangement of their carbon atoms is superimposable on a fragment of an FCC diamond lattice.

The state of the art differentiates lower and higher diamondoids.

Hereby, “lower diamondoids” are understood to be adamantane, diamantane and triamantane, as well as all substituted and unsubstituted derivatives of these compounds. These lower diamondoids occur neither in different isomeric forms nor are they chiral. This is what distinguishes them from higher diamondoids.

The term “higher diamondoids” refers to all substituted and unsubstituted tetramantanes, pentamantanes, hexamantanes, heptamantanes, octamantanes, nonamantanes, decamantanes, undecamantanes, etc., as well as their isomers and stereoisomers.

Adamantane is the smallest member of the diamondoid series and consists of a single cage structure of the diamond crystal lattice. Diamantane consists of two adamantane subunits which are condensed with each other via the front sides, triamantane of three, tetramantane of four, etc. Whilst there is only one isomeric form of adamantane, diamantane and triamantane, there are already four isomers of tetramantane of which two form an enantiomeric pair, i.e. four different possible arrangements of the adamantane subunits. The amount of possible isomers increases non-linearly with each higher member of the diamondoid series.

From five adamantane subunits onwards, i.e. from pentamantane, structures with different molecular formulae at a given amount of adamantane subunits exist. These different molecular formulae result due to particular arrangements of the adamantane subunits.

The four different tetramantane structures are iso-tetramantanes [1(2)3], anti-tetramantanes [121] and the two enantiomeric skew-tetramantanes [123]. All four tetramantanes possess the molecular formula C₂₂H₂₈ (molecular weight 292).

Hereby, the naming of these diamondoids follows a convention which was introduced by Balaban et al. in “Systematic Classification and Nomenclature of Diamond Hydrocarbons-I”, Tetrahedron, 1978, 34, 3599-3609

There are ten different pentamantanes, of which nine have the molecular formula C₂₆H₃₂ (molecular weight 344). Among these nine pentamantanes, there are three enantiomeric pairs represented generally by [12(1)3], [1234] and [1213], wherein the remaining three pentamantanes are represented by [12(3)-4], [1212] and [1(2,3)-4]. Furthermore, there also exists a pentamantane [1231] represented by the molecular formula C₂₆H₃₀ (molecular weight 330).

Hexamantanes can occur in thirty-nine possible structures, of which twenty eight have the molecular formula C₃₀H₃₆ (molecular weight 396). Ten hexamantanes have the molecular formula C₂₉H₃₄ (molecular weight 382), and the remaining hexamantane [13212] has the molecular formula C₂₆H₃₀ (molecular weight 342).

Heptamantanes are postulated to exist in 160 possible structures, of which 85 comprise the molecular formula C₃₄H₄₀ (molecular weight 448) and, of those, seven are achiral. Of the remaining heptamantanes, 67 have the molecular formula C₃₃H₃₈ (molecular weight 434), six have the molecular formula C₃₂H₃₆ (molecular weight 420) and the remaining two have the molecular formula C₃₀H₃₄ (molecular weight 394).

Octamantanes comprise eight adamantane subunits and exist in structures with five different molecular weights. Among the octamantanes, 18 have the molecular formula C₄₃H₃₈ (molecular weight 446). Octamantanes also exist with the molecular formula C₃₈H₄₄ (molecular weight 500), C₃₇H₄₂ (molecular weight 486), C₃₆H₄₀ (molecular weight 472), and C₃₃H₃₆ (molecular weight 432).

Concerning nonamantanes, there are six families of different molecular weights with the following molecular formulas: C₄₂H₄₈ (molecular weight 552), C₄₁H₄₆ (molecular weight 538), C₄₀H₄₄ (molecular weight 524), C₃₇H₄₀ (molecular weight 484) and C₃₄H₃₆ (molecular weight 444).

Concerning decamantanes, there are seven families with different molecular weights. Among the decamantanes, there is a single compound having the molecular formula C₃₅H₃₆ (molecular weight 456), which is structurally compact compared to the other decamantanes. The other decamantane families have the following molecular formulas: C₄₆H₅₂ (molecular weight 604), C₄₅H₅₀ (molecular weight 590), C₄₄H₄₈ (molecular weight 576), C₄₂H₄₆ (molecular weight 550), C₄₁H₄₄ (molecular weight 536) and C₃₈H₄₀ (molecular weight 496).

Concerning the undecamantanes, there are families with eight different molecular weights. Among the undecamantanes, there are two compounds having the molecular formula C₃₉H₄₀ (molecular weight 508), which are structurally compact compared to the other undecamantanes. The other undecamantane families have the following molecular formulas: C₄₁H₄₂ (molecular weight 534); C₄₂H₄₄ (molecular weight 548), C₄₅H₄₈ (molecular weight 588), C₄₆H₅₀ (molecular weight 602), C₄₈H₅₂ (molecular weight 628), C₄₉H₅₄ (molecular weight 642) and C₅₀H₅₆ (molecular weight 656).

Diamondoids occur naturally in crude oil, natural gas and other materials that are rich in hydrocarbon compounds. Naturally occurring diamondoids also comprise alkyl-substituted compounds.

Diamondoids are of interest as starting materials for microelectronics, pharmacy, nanotechnology, and material sciences. Potential applications of the diamondoids and their derivatives are, for instance, the production of thermally stable plastics, coatings with tailored conductivities for LEDs and transistors, nanoelectronics as well as application in pharmaceuticals against viral and neurodegenerative diseases.

Hereby, diamantanes with hydroxy groups, carbonyl groups, carboxyl groups, amino groups and/or aminocarbonyl groups are of particular interest as they are important intermediates for the production of oligomeric and polymeric diamantanes. Furthermore, the aforementioned functional groups are suitable for being substituted by other functional groups in such a way that substituted diamanatanes represent important starting materials for the production of further substituted diamantanes.

It is known to persons skilled in the art that the skeleton of diamondoids comprises different carbon atoms: secondary (2° or C-2), tertiary (3° or C-3) and quaternary (4° or C-4) carbon atoms, wherein quaternary carbon atoms exist from triamantane onwards. Quarternary carbon atoms are unable undergo substitution reactions. Chemical reactions can only occur at secondary and tertiary carbon atoms of diamondoids. It is to be noted that some of the tertiary or secondary carbon atoms, respectively, are equivalent. Derivatives which are substituted at these equivalent secondary or tertiary, carbon atoms are identical.

There are three reaction mechanisms for the derivatization of diamondoids: nucleophilic (S_{N}1 type) and electrophilic (S_{E}2 type) substitution reactions and free radical reactions. These are described, for example, in EP 1,453,777 B1.

There are already several works concerning the introduction of different functional groups into diamondoids, by way of example:
1. L. Vodicka, J Janku, J Burkhard: “Synthesis of diamantanecarboxylic acids with the carboxy groups bonded at tertiary carbon atoms”. Collect Czech Chem Commun 1983, 48, 1162-1172
2. L Vodicka, J Burkhard, J Janku: “Preparation of diamantanecarboxylix acids with carboxyl groups on one secondary and one tertiary carbon atom”. Collect Czech Chem Commun 1986, 51, 867-871
3. J Burkard, J Janku, P Zachar, L Vodicka: “Hydroxydiamantanecarboxylic and diamantanecarboxylic acids”. Sb Vys Sk Chem Techn 1989, D57, 5-16
4. J Burkard, J Janku, L Vodicka: “Diamantanecarboxylic acids with carboxyls on tertiary carbon atoms of the diamantane skeleton” Sb Vys Sk Chem Techn 1983, D47, 73-99
5. J Janku, J Burkard, L Vodicka: “Preparation and isomerization of hydroxydiamantanecarboxylic acids”. Sb Vys Sk Chem Techn 1984, D49, 25-38
6. L Vodicka, S D Isaev, J Burkhard, J Janku: “Synthesis and reactions of hydroxydiamantanones”. Collect Chem Czech Commun 1984, 49, 1900-1906

The methods described here yield product mixtures.

In M Padmanaban, S Chakrapani, G Lin, T Kudo, D Parthasarathy, C Anyadiegwu, C Antonio, R Dammel, S Liu, F Lam, T Maehara, F Iwasaki, M Yamaguchi: “Novel Diamantane Polymer Platform for Resist Applications”, J Photopol Sci Technol 2007, 20, 719-728, the production of methacrylic acid derivatives of the diamantane is described. The synthesis of 3-alkyl-3-diamantyl-methacrylates is realized by first reacting diamantane with concentrated sulfuric acid to 3-diamantanone. Subsequently, 3-diamantanone is reacted with an alkyl-Grignard compound and afterwards with methacryloyl chloride. In this reaction sequence, it is not possible to introduce substituents at different carbon atoms of the diamantane skeleton and the yield of the respective methacrylic acid derivative is only 40%. Furthermore, this work describes the production of 9-hydroxy-4-diamantyl-methacrylate. For that purpose, diamantane is reacted with chlorosulfuric acid and sulfuric acid to the 4,9-diamantane diol. Subsequently, equimolar amounts of diol and methacryloyl chloride react to 9-hydroxy-4-diamantyl-methacrylate. The yield is, however, only 5%.

WO 00/342141 A1 describes aminohydroxy adamantane derivatives and their use as dipeptidyl peptidase IV inhibitors, for instance for the treatment of diabetes. However, in the case of two identical functional groups, the presented methods for producing these adamantane derivatives do not allow for the conversion of just one functional group into another functional group.

WO 2007/069656 A1 describes a method for producing a polymerizable hydroxydiamantylester compound. Hereby, a 4,9-diamantanediol compound is produced by dihalogenating and subsequent hydrolyzing with water. Subsequently, the 4,9-diamantanediol compound is esterified with a mixture of unsaturated carboxylic acid and an anhydride of an unsaturated carboxylic acid in the presence of a polymerization inhibitor and an acid catalyst.

Aminoadamantanecarboxylic acids are described In WO 2006/010362 A1 as well as in L Wanka, C Cabrele, M Vanejews, P R Schreiner: “γ-Aminoadamantanecarboxylic acids through direct C—H bond amidations”, Eur J Org Chem 2007, 1474-1490. These compounds are produced by direct amide formation with the help of a Ritter reaction in nitrating acid. Prior halogenation of the adamantane is not necessary.

The Ritter reaction is known to persons skilled in the art. It allows for the reaction of alkenes, secondary or tertiary alcohols with nitriles to amides. If halogen nitriles are used, a halogen alkanoyl group is introduced which is subsequently cleaved off again through a reaction with thiourea. In this way, tertiary alcohols, for instance, can be converted into the corresponding tertiary amino compounds. For this, refer to A Jirgensons, V Kauss, I Kalvinsh, M R Gold: “A Practical Synthesis of tert-Alkylamines via the Ritter Reaction with Chloroacetonitrile”, Synthesis 2000, 12, 1709-1712.

US 2002/0177743 A1 describes derivatives of higher diamondoids which comprise one or two polymerizable functional groups, as well as intermediates which are useful for the synthesis of the polymerizable higher diamondoids. Among both the intermediates and the polymerizable higher diamondoids are, for instance, derivatives with hydroxyl, amino and carbonyl functions. Production methods for substituted higher diamondoids are also provided. However, all methods provided by US 2002/0177743 A1 use mixtures of starting materials, so that product mixtures are yielded. The methods provided in this patent specification would also yield product mixtures by using pure starting materials, and it is not possible to introduce determined functional groups regio- and/or stereoselectively.

Similar functionalized higher diamondoids to those of US 2002/0177743 A1 are also described in EP 1 453 777 B1. EP 1 453 777 B1 indicates that diamondoids in nucleophile substitutions react according to S_{N}1, wherein stable diamondoid carbocations are yielded. Such stable diamondoid carbocations are suitable for being generated, for instance, from hydroxylated diamondoids.

In DE 10 2005 058 357 A1, as well as in N A Fokina, B A Tkachenko, A Merz, M Serafin, J E P Dahl, R M K Carlson, A A Fokin, P R Schreiner: “Hydroxy derivatives of diamantane, triamantane, and [121]tetramantane: selective preparation of bis-apical derivatives.” Eur J Org Chem 2007, 4738-4745, how diamondoids react to the corresponding nitroxylated derivatives is described. The nitroxy groups are suitable for being subsequently converted into hydroxy groups.

It is known to persons skilled in the art that the introduction of hydroxy groups is advantageous, because the hydroxy group is suitable for being converted into a large number of further functional groups, for instance into amino and carbonyl groups.

However, none of the publications mentioned here discloses a method which would enable the targeted conversion of only one out of several hydroxy groups into another functional group and, thus, prevent the formation of product mixtures or at least reduce the amount of possible different reaction products of polyhydroxylated diamondoids.

In the case of many applications, for instance, in microelectronics, material sciences, and pharmacy, it is desirable or even required to use pure substances and not substance mixtures and that the substance mixtures are halogen-free. The separation of substance mixtures is, however, often very time-consuming and expensive. Furthermore, many of the methods known so far require the halogenation of the diamondoid skeleton for producing substituted diamondoids. The halogenation is mostly a bromination. Thus, production methods which yield the smallest amount of different products possible and which possibly work without bromination (or in general, without halogenation) are desirable. Those methods which yield one single substance as the main product are preferable.

In contrast, the invention at hand provides, for the first time, derivatives of diamondoids comprising two hydroxy groups, of which one is masked by a protective group. Thus, the derivatives of diamondoids according to the present invention allow the selective conversion of a hydroxy group into another functional group, wherein the second masked hydroxy group is subsequently released again and suitable for then being converted optionally into another functional group as well.

### Aim

The aim of the invention at hand is to provide functionalized diols of diamondoids in which one of the two hydroxy groups is masked by a protective group, as well as methods for their production.

### Achievement of this Aim

The aim of providing functionalized diols of diamondoids in which one of the two hydroxy groups is masked by a protective group is achieved according to the present invention by compounds according to formula (I)

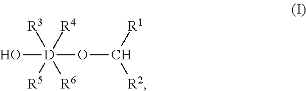

wherein
   D represents a diamondoid,
   R¹ stands for a linear or branched alkyl group —CnHₚX_{q},
      wherein n is a natural number from 1 to 25,
   p is an integer between 0 and 50 and q is a natural number between 1 and 51,
   and the sum of q and p is (2n+1),
R² stands for hydrogen or a linear or branched alkyl group —CₘHᵣXs,
   wherein m is a natural number from 1 to 25,
   r and s are integers between 0 and 51,
   and the sum of r and s is (2 m+1),
X stands for a halogen selected from fluorine, chlorine or bromine,
R³, R⁴, R⁵ and R⁶ represent, independently of one another:
   hydrogen,
   a linear or branched alkyl group having 1 to 20 carbon atoms,
   a cyclic alkyl group having 3 to 20 carbon atoms,
   an aryl group having 6 to 18 carbon atoms,
   a heteroaryl group having 5 to 18 ring atoms, of which 1 or 2 atoms are heteroatoms selected from nitrogen, oxygen, sulfur, and the remaining ring atoms are carbon atoms.

Surprisingly, it has been found that groups according to formula (II)

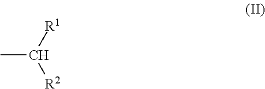

are suitable as protective groups for masking hydroxy groups. Hereby R¹ and R² are defined as described above.

The functionalized diols of diamondoids according to the present invention are explained hereinafter, wherein the invention comprises individually and in combination with one another all the preferred embodiments presented below.

In the functionalized diols of diamondoids according to the present invention one of two hydroxy groups is masked by a protective group according to formula (II), namely in the form of an ether. Thus, functionalized diols of diamondoids according to the present invention according to formula (I) are hereinafter also referred to as “diamondoid monoether” or in brief “monoether”.

The term “diamondoid” refers to the substituted and unsubstituted cage-like compounds of the adamantane series which were described at the beginning. Thus, it also comprises, by way of example but not exhaustively, substituted and unsubstituted adamantane, diamantane, triamantane, tetramantane, pentamantane, hexamantane, heptamantane, octamantane, nonamantane, decamantane, undecamantane, etc. Substituted diamondoids carry substituents R³, R⁴, R⁵ and R⁶ according to formula (I).

Furthermore, “diamondoid” or “diamondoids”, respectively, comprise homologous, analogous and isomeric compounds.

Hereby, “homologous” diamondoids are understood to be a series of compounds which can be represented through a general molecular formula and in which one compound of the series is formed out of the previous substance by “adding” a further “chain link”. In the case of homologous diamondoids, the chain link is formally a C₄H₄ unit. In this way, diamantane is formally obtained from adamantane by adding a C₄H₄ unit, triamantane by adding a C₄H₄ unit to that, etc.

“Analogous” diamondoids, in contrast, are compounds with identical amount of adamantane subunits, but different molecular formula and different molecular weights. Analogues exist, as explained in the beginning, in diamondoids which comprise at least five adamantane subunits, namely from pentamantane onwards.

“Isomeric” diamondoids in the sense of the present invention comprise the same molecular formula, but a different arrangement of the adamantane subunits and/or the substituents R³, R⁴, R⁵ and R⁶.

“Lower diamondoids” are understood to be all substituted and/or unsubstituted adamantanes, diamantanes and triamantanes and their derivatives and isomers.

Substituted and/or unsubstituted tetramantanes, pentamantanes, hexamantanes, heptamantanes, octamantanes, nonamantanes, decamantanes, undecamantane (eleven adamantane subunits) and all further diamondoids with more than eleven adamantane subunits, as well as their derivatives, analogues and isomers are referred to as “higher diamondoids”.

As described at the beginning, there is only one isomeric form of unsubstituted adamantane, diamantane and triamantane, respectively, but there are already four isomers of the unsubstituted tetramantane of which two form an enantiomeric pair, i.e. four different possible arrangements of the adamantane subunits.

If the lower diamondoids adamantane, diamantane and triamantane are substituted they also occur in different isomers, provided that at least one of the four substituents R³, R⁴, R⁵ and R⁶ according to the definition above is not hydrogen. From tetramantane onwards, different isomers are possible both in the unsubstituted and the substituted diamondoid, respectively.

The invention at hand refers to all lower, higher, substituted, unsubstituted, homologous, analogous and/or isomeric diamondoids according to the definition above.

According to the definition above, substituents R³, R⁴, R⁵ and R⁶ are bound to the functionalized diols of diamondoids according to the present invention. According to the definition above, these substituents stand for hydrogen, a linear or branched alkyl group, a cyclic alkyl group, an aryl group or a heteroaryl group independently of one another.

If R³, R⁴, R⁵ and/or R⁶ is or are a linear or branched alkyl group, each of these alkyl groups independently of one another contains 1 to 20 carbon atoms. These alkyl groups are for example methyl, ethyl, n-propyl, isopropyl, 1-butyl, 2-butyl, tert-butyl, 1-pentyl, 2-pentyl, 3-pentyl, 3-methylbutyl, 2,2-dimethylpropyl, and also all the isomers of hexyl, heptyl, octyl, nonyl, decyl, undecyl, dodecyl.

If R³, R⁴, R⁵ and/or R⁶ is or are a cyclic alkyl group having 3 to 20 carbon atoms, this is preferably selected from cyclopentyl, cyclohexyl, cycloheptyl.

If R³, R⁴, R⁵ and/or R⁶ is or are an aryl group having 6 to 18 carbon atoms, this is selected from phenyl, naphthyl, anthracenyl, phenanthrenyl, tetracenyl.

Furthermore, R³, R⁴, R⁵ and/or R⁶ may also be a heteroaryl group having 5 to 18 ring atoms, of which 1 or 2 atoms are heteroatoms selected from nitrogen, oxygen and sulfur, wherein the remaining ring atoms are carbon atoms. The heteroaryl group is selected from furanyl, benzofuranyl, isobenzofuranyl, pyrrolyl, indolyl, isoindolyl, thiophenyl, benzothiophenyl, benzo[c]thiophenyl, imidazolyl, benzimidazolyl, pyrazolyl, indazolyl, oxazolyl, benzoxyzolyl, isoxazolyl, benzisoxazolyl, thiazolyl, benzothiazolyl, pyridinyl, quinolinyl, isoquinolinyl, pyrazinyl, quinoxalinyl, acridinyl, pyrimidinyl, quinazolinyl, pyridazinyl, cinnolinyl.

R¹ is a linear or branched alkyl group —CnHₚX_{q}. Herein, n is a natural number from 1 to 25, p is an integer between 0 and 50 and q is a natural number between 1 and 51. The sum of p and q is (2n+1).

R² is a hydrogen or a linear or branched alkyl group —CₘHᵣXₛ. Herein, m is a natural number from 1 to 25, and r and s are integers between 0 and 51. The sum of r and s is (2 m+1).

The halogen atom X in R¹ and R² is selected from fluorine, chlorine and bromine. R¹ is thus formally an alkyl group —CₙH₂ₙ₊₁, in which at least one and at most all hydrogen atoms are replaced by a halogen atom X. R² is an alkyl group —CₘH₂ₘ₊₁, in which none to all hydrogen atoms are replaced by a halogen atom X, provided that R² does not stand for a hydrogen atom.

If R1 and/or R2 are at least monohalogenated and at most perhalogenated alkyl groups —CₙH₂ₙ₊₁ or —CmH₂ₘ₊₁, then these are for example the methyl, ethyl, n-propyl, isopropyl, 1-butyl, 2-butyl, tert-butyl, 1-pentyl, 2-pentyl, 3-pentyl, 3-methylbutyl, 2,2-dimethylpropyl groups halogenated according to the definition above, and also all the isomers of the hexyl, heptyl, octyl, nonyl, decyl, undecyl and dodecyl groups halogenated according to the above definition. Analogously, R² may also be the corresponding non-halogenated alkyl groups.

The sum of n and m is in this case a natural number between 1 and 25. According to the definitions of R¹, R², m, n, p, q, r and s given here, the protective group —CHR¹R² according to the present invention accordingly contains at least two and at most 26 carbon atoms.

R¹ and R² are of course only suitable for being branched when n or m, respectively, is at least equal to 3.

In a preferred embodiment, the halogen atom X is fluorine.

In a further preferred embodiment, the residues R³ and R⁴ are in each case a hydrogen atom and the residues R⁵ and R⁶ are in each case a linear or branched alkyl group having 1 to 20 carbon atoms.

Furthermore, preference is given to those embodiments in which R³ and R⁴ stand for hydrogen and R⁵ and R⁶ are identical. Very particular preference is given to those functionalized diols in which R³ and R⁴ stand for hydrogen and R⁵ and R⁶ are identical and R⁵ and R⁶ stand for a linear or branched alkyl group having 1 to 20 carbon atoms.

In a further preferable practical embodiment, the functionalized diols of diamondoids according to the present invention are compounds in which the protective group according to formula (II)

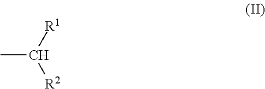

comprises a total of 2 to 12 carbon atoms.

In a further preferable practical embodiment, the protective group according to formula (II) comprises a total of 2 to 12 carbon atoms, and the halogen atom X is fluorine.

Most preferred are protective groups —CHR¹R² selected from —O—CH₂—CF₃, —O—CH₂—CF₂H, —O—CH₂—CF₂—CF₃, —O—CH₂—CF₂—CF₂H, —O—CH—(CF₃)₂, —O—CH₂—(CF₂)₂—CF₃, —O—CH₂—(CF₂)₂—CF₂H, —O—CH₂—(CF₂)₃—CF₃, —O—CH₂—(CF₂)₃—CF₂H.

The functionalized diols of diamondoids according to the present invention are produced by reacting a diamondoid diol (III) with a halogenated alcohol (IV) in the presence of a Brönsted or Lewis acid as catalyst as follows:

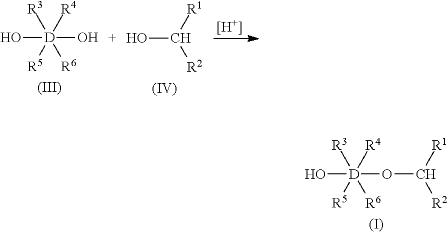

Hereby, R¹, R², R³, R⁴, R⁵ and R⁶ are as defined above.

The reaction of diamondoid diol (III) and alcohol (IV) to the monoether (I) according to the formula above is a S_{N}1 reaction.

The method according to the present invention for producing functionalized diols of diamondoids comprises the following steps:
a) mixing a diamondoid diol DR³R⁴R⁵R⁶(OH)₂ with a halogenated alcohol CHOHR¹R², wherein D, R¹, R², R³, R⁴, R⁵ and R⁶ are as defined above,
b) adding a catalyst acid,
c) stirring at −20° C. to 100° C.,
d) ending the reaction by adding an amine or water,
e) isolating the crude end product,
f) purifying the crude end product.

The mixing ratio of diamondoid diol and halogenated alcohol plays no role for the success of the reaction, i.e. for the successful formation of the monoether according to the present invention. The mixture of diamondoid diol and halogenated alcohol may accordingly be concentrated or diluted solutions or also suspensions.

Use is preferably made of fluorinated alcohols, for example HO—CH₂—CF₃, HO—CH₂—CF₂H, HO—CH₂—CF₂—CF₃, HO—CH₂—CF₂—CF₂H, HO—CH—(CF₃)₂, HO—CH₂—(CF₂)₂—CF₃, HO—CH₂—(CF₂)₂—CF₂H, HO—CH₂—(CF₂)₃—CF₃, HO—CH₂—(CF₂)₃—CF₂H.

The use of fluorinated alcohols is particularly advantageous due to their low boiling points compared to the non-fluorinated analogs.

A Brönsted or Lewis acid is selected as the catalyst acid. Suitable catalyst acids are for example CF₃SO₃H and p-toluenesulfonic acid. Advantageously 10⁻⁵ vol % to 10 vol % of catalyst acid, preferably approximately 0.1 vol %, is added, relative to the volume of the solution of the diamondoid diol.

Following the addition of the catalyst acid, the reaction mixture is stirred at around −20° C. to 100° C. Preference is given to a reaction temperature from 25° C. to approximately 40° C. The reaction time to be selected for a given combination of diamondoid diol and halogenated alcohol can easily be determined by persons skilled in the art by means of routine experiments and without leaving the scope of protection of the patent claims. Hereby, halogenated alcohols are compounds HO—CHR¹R², which, according to the definition above of the protective group —CHR¹R², comprise at least one halogen atom X, selected from fluorine, chlorine and bromine. In general, higher diamondoids are more reactive than their lower homologues and analogues as regards the required reaction time and temperature. Furthermore, OH groups are more reactive in medial positions of the diamondoid than those in apical positions.

The etherification reaction between diamondoid diol and halogenated alcohol can be ended by adding an amine or by adding water. By adding an amine, the catalyst acid is neutralized. Suitable amines are for example triethylamine and diisopropylethylamine (DIPEA). If the reaction is ended by adding an amine, the crude end product is advantageously isolated by removing the solvent.

Alternatively, the reaction can be stopped by adding water. If the reaction is ended by adding water, the crude end product is isolated from the reaction mixture by extraction, for example with chloroform.

It is preferable to end the reaction by adding an amine, since in this case higher yields of the monoether are achieved than when the reaction is stopped by adding water. Furthermore, more operating steps (extraction, removal of the extracting agent) are necessary if the reaction is ended by adding water.

The purification of the crude end product takes place by means of methods known to persons skilled in the art, for example by means of column chromatography. Purification methods for diamondoid derivatives are known to persons skilled in the art and can be used without leaving the scope of protection of the patent claims.

The diamondoid monoethers according to the present invention can be used for the targeted production of further diamondoid derivatives. Particularly advantageous in this case is the Ritter reaction of the free hydroxy group with a halogenated carboxylic acid nitrile to form an amide according to

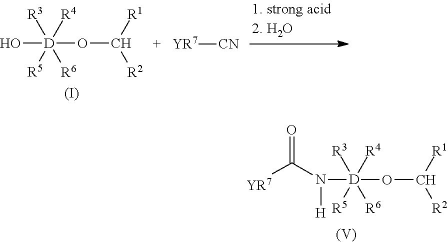

Hereby, R¹ to R⁶ are as defined above, R⁷ stands for a linear, branched or cyclic alkylene group —CₜH₂ₜ—, wherein t is a natural number from 1 to 11, and Y is a halogen atom selected from fluorine, chlorine, bromine, iodine.

In order to carry out the Ritter reaction, the monoether (I) is dissolved in glacial acetic acid and is mixed with the halogenated nitrile at room temperature. Subsequently, concentrated sulfuric acid is added. The reaction is ended by adding water. Suitable nitriles for the Ritter reaction and also the necessary reaction conditions are known to persons skilled in the art and can be used without leaving the scope of protection of the patent claims.

The nitriles are selected for example from halogenated acetonitrile, propionitrile, butyronitrile, valeronitrile, capronitrile, heptanoic acid nitrile, octanoic acid nitrile, nonanoic acid nitrile, decanoic acid nitrile, undecanoic acid nitrile, lauric acid nitrile and cyclopropanecarboxylic acid nitrile. Use is preferably made of chlorinated nitriles.

The amides of the monoethers which are produced in this reaction will be referred to hereinafter as “monoether amides”.

The monoether amides formed in this way can be used to produce aminoalcohols or aminocarboxylic acids of the diamondoids.

In order to produce amino alcohols, in the first step a hydroxy group of a diamantane diol is masked with a protective group —CHR¹R².

This monoether is reacted as described above in a Ritter reaction to form the corresponding monoether amide. Subsequently, the alkyloxy group —O—CHR¹R² of the monoether amide reacts with trifluoroacetic acid. By way of example, this is shown in practical embodiment 6 for the production of 4-trifluoroacetoxy-9-(2-chloroacetylamino)-diamantane 8 from 2-chloro-N-[9-(2,2,2-trifluoroethoxy)-diamantane-4-yl-acetamide 5.]

Finally, the two alkanoyl groups (—OC—CF₃ and —COR⁷Y) are removed by heating with thiourea, ethanol and glacial acetic acid, and the reaction solution is then made alkaline by using an aqueous alkaline solution, for example NaOH or KOH. Finally, the crude amino alcohol is isolated from the reaction mixture and purified. By way of example, this is shown in practical embodiment 7 for the production of 4-amino-diamantane-9-ol 9 from 4-trifluoroacetoxy-9-(2-chloroacetylamino)-diamantane 8.

The corresponding aminocarboxylic acid can be produced from this amino alcohol. For that purpose, the amino alcohol is reacted with a mixture of oleum and 100% formic acid at temperatures between −20° C. and 0° C. Subsequently, an alkaline solution is added, for example NaOH or KOH, in order to precipitate out the crude aminocarboxylic acid salt. The aminocarboxylic acid salt is then cleaved off, purified and dried. Subsequently, it is reacted with thionyl chloride/methanol to form the corresponding aminocarboxylic acid methyl ester. The latter is then isolated and purified. By way of example, this is shown in practical embodiment 10 for the production of 4-amino-9-diamantanecarboxylic acid methyl ester 11 from 4-aminodiamantane-9-ol 9. By addition of a strong mineral acid and heating, the ester can be saponified to carboxylic acid hydrochloride which is finally cleaved off, purified and dried, as shown in practical embodiment 11 for the production of 4-amino-9-diamantanecarboxylic acid hydrochloride 12 from 4-amino-9-diamantanecarboxylic acid methyl ester 11.

Alternatively, aminocarboxylic acids of the diamandoids can be produced by heating a monoether amide with thiourea, ethanol and glacial acetic acid. The reaction solution is then diluted with water and made alkaline by using an alkaline solution, for example NaOH or KOH, then the monoether amine obtained is isolated and purified. This is shown by way of example in practical embodiment 4 for the production of 9-(2,2,2-trifluoroethoxy)-diamantane-4-amine 6 from 2-chloro-N-[9-(2,2,2-trifluoroethoxy)-diamantane-4-yl]-acetamide 5. Subsequently, the monoether amine is reacted with a mixture of concentrated sulfuric acid and 100% formic acid at temperatures between −20° C. and 0° C. Subsequently, the mixture is poured onto ice and the aminocarboxylic acid is isolated and purified. This is shown by way of example in practical embodiment 5 for the reaction of 9-(2,2,2-trifluoroethoxy)-diamantane-4-amine 6 to 4-amino-9-diamantanecarboxylic acid 7.

Hereby, preference is given to producing aminocarboxylic acids by converting the corresponding amino alcohol into the methyl ester and subsequent saponification of the ester with a strong mineral acid (refer to practical embodiments 10 and 11).

Amino alcohols and aminocarboxylic acids of the diamondoids are particularly suitable for introducing other functional groups in a targeted manner. This is described hereinafter for the production of nitro alcohols of the diamondoids from the respective aminoalcohols.

By way of example, the corresponding nitro alcohols can be produced from the amino alcohols of the diamondoids, for example by reaction with meta-chloroperbenzoic acid (mCPBA). This is shown in practical embodiment 8 for the oxidation of 4-aminodiamantane-9-ol) to 4-nitrodiamantane-9-ol 10.

Further possibilities for converting amino, hydroxy and carboxyl groups into other functional groups are known to persons skilled in the art.

The protective group —CHR¹R² according to the present invention can be converted back into an OH group by treatment with a strong acid, for example CF₃COOH or H₂SO₄, followed by neutralization with an alkaline solution, e.g. NaOH or KOH.

By means of the monoethers of diamondoids described in the present invention, it is possible to produce medically relevant diamondoids, for example, in a targeted manner and in a higher yield and purity than before.

Furthermore, the monoethers according to the present invention allow for the control of the electron exit behavior of the diamondoids by a targeted introduction of groups which emit electrons well or badly. Thus, the monoethers according to the present invention and the method for their production are helpful for the production of diamondoid derivatives for electronic components.

Furthermore, aminocarboxylic acids and amino alcohols of the diamondoids allow for the introduction of functional groups for the polycondensation and also for the production of peptides.

### EMBODIMENTS

### Embodiment 1

### Production of 3-(2,2,2-trifluoroethoxy)-adamantane-1-ol

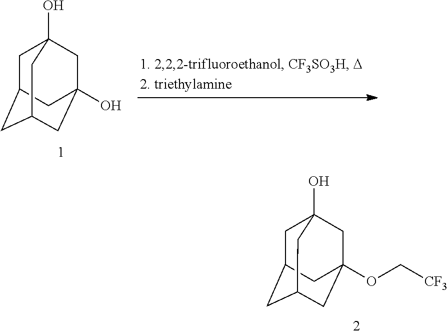

0.3 mmol (50 mg) 1,3-adamantanediol 1 with a purity of >99% was dissolved in 40 ml 2,2,2-trifluoroethanol and 10 μL CF₃SO₃H was added at 40° C. in a water-bath. After stirring for 2.5 h at the same temperature, the acid was neutralized with 20 μL triethylamine and the solvent removed to dryness. The oily raw product was separated over silica gel with ethylacetate:hexane 8:2 as mobile solvent. Thereby, 56 mg (75%, R_{f}=0.44) of the pure monoether 2 (slowly crystallizing oil) was obtained.

Spectroscopic data for 3-(2,2,2-trifluoroethoxy)-adamantane-1-ol:

¹H-NMR (400 MHz, CDCl₃) δ: 3.80 (2H, q, J=8.8 Hz), 2.35 (2H, br s), 1.78-1.62 (10H, m), 1.59-1.47 (3H, m)

¹³C-NMR (100 MHz, CDCl₃) δ: 124.2 (q, J=277.7 Hz), 76.2, 70.4, 59.4 (q, J=34.2 Hz), 48.7, 44.0, 39.8, 34.7, 30.9

¹⁹F-NMR (376 MHz, CDCl₃) δ: −74.55

IR data (KBr): 3350.3, 2919.9, 2858.7, 1456.3, 1352.5, 1275.8, 1171.1, 1151.7, 1131.2, 1111.4, 1094.5, 1014.2, 963.0 cm⁻¹

Melting point: 78° C.

HRMS: calculated for C₁₂H₁₇F₃O₂: 250.1181. found: 250.1182

C, H, N: calculated for C₁₂H₁₇F₃O₂ (250, 26): C, 57.59, H, 6.85. found: C, 57.43, H, 6.83

### Embodiment 2

### Production of 4-(2,2,2-trifluoroethoxy)-diamantane-9-ol

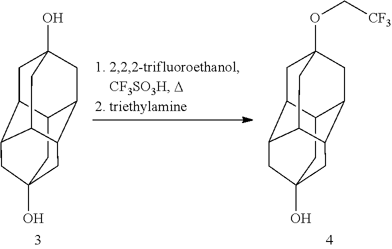

2.5 mmol (551 mg) 4,9-diamantanediol 3 with a purity of >99% was dissolved in 100 ml 2,2,2-trifluoroethanol and 50 μL CF₃SO₃H was added at 40° C. in a water-bath. After stirring for 4 h at the same temperature, the acid was neutralized with 100 μL triethylamine and the solvent removed to dryness. The colorless, solid, raw product was separated over silica gel with ethylacetate:hexane 8:2 as mobile solvent. Thereby, 426.3 mg (56%, R_{f}=0.36) of the pure monoether 4 was obtained as colorless solid. Furthermore, 82.5 mg (9%, R_{f}=0.73) of the pure diether and 165.9 mg (30%, R_{f}=0.15) of the starting material were obtained.

Spectroscopic data for 4-(2,2,2)-trifluoroethoxy-diamantane-9-ol:

¹H-NMR (400 MHz, CDCl₃) δ: 3.79 (2H, q, J=8.8 Hz), 2.01-1.90 (6H, m), 1.83-1.72 (12H, m), 1.33 (1H, OH, br s)

¹³C-NMR (100 MHz, CDCl₃) δ: 124.3 (q, J=277.7 Hz), 73.1, 67.1, 59.3 (q, J=35.2 Hz), 44.5, 40.3, 38.8, 38.3

¹⁹F-NMR (376 MHz, CDCl₃) δ: −74.55

IR data (KBr): 3277.7, 2925.2, 2903.5, 2888.6, 2854.7, 1444.5, 1417.6, 1349.6, 1301.0, 1278.4, 1146.2, 1103.7, 1007.8, 962.0, 836.0, 663.0 cm⁻¹

Melting point: 149° C.

HRMS: calculated for C₁₆H₂₁F₃O₂: 302.1494. found: 302.1489

C, H, N: calculated for C₁₆H₂₁F₃O₂ (302, 32): C, 63.56, H, 7.00. found: C, 63.30, H, 6.90

### Embodiment 3

### Production of 2-chloro-N-[9-(2,2,2-trifluoroethoxy)-diamantane-4-yl]-acetamide

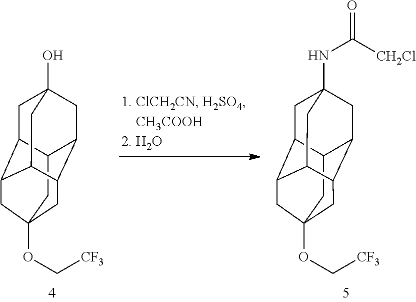

6.48 mm (1.96 g) 4-(2,2,2)-trifluoroethoxy diamantane-9 of 4 with a purity of >99% was dissolved in 30 ml glacial acetic acid and reacted at room temperature with 9 mL (0.1 mol) chloroacetonitrile. Subsequently, 4.5 ml conc. sulfuric acid was added under stirring and ice bath cooling. The clear solution was stirred subsequently for 30 min in ice-bath and another 17 h at room temperature. By addition of 120 ml distilled water, a colorless precipitate was obtained which was dissolved by extraction 4 times with respectively 50 ml CHCl₃. The combined organic phases were washed twice with 75 ml distilled water and subsequently dried over Na₂SO₄. After filtering off the desiccant and removal of the solvent, a beige, solid crude product was obtained. The raw product was separated over silica gel with CH₂Cl₂:ethylacetate 85:15 as mobile solvent. Thereby, 1.01 g (R_{f}=0.57) of the pure product was obtained. After further separation of a mixed fraction over silica gel with ether:pentane 2:1 another 97.4 mg (R_{f}=0.38) of the product was obtained. In total, 1.11 g (overall yield 45%) of the 2-chloro-N-[9-(2,2,2-trifluoroethoxy)-diamantane-4-yl]-acetamide 5 was obtained in this way. Furthermore, it was possible to isolate the following by-products via different separation methods:

4-acetoxy-9-(2,2,2)-trifluoroethoxy-diamantane: 108 mg (5%) via the CH₂Cl₂:ethylacetate separation (R_{f}=0.69)

Bis-4,9-acetoxy-diamantane: 19 mg (1%) via the CH₂Cl₂:ethylacetate separation (R_{f}=0.65)

2-chloro-N-[9-acetoxy-diamantane-4-yl]-acetamide: 237 mg (11%) via the CH₂Cl₂:ethylacetate separation (R_{f}=0.41)

4-(2,2,2)-trifluoroethoxy-diamantane-9-ol: 105 mg (5%) of the starting material via the CH₂Cl₂:ethylacetate separation (R_{f}=0.26)

2-chloro-N-[9-(2-chloro-acetylamino)-diamantane-4-yl]-acetamides: 116 mg (5%) via the CH₂Cl₂:ethylacetate separation (R_{f}=0.24), as well as further purification over silica gel with ethyl acetate as mobile solvent (with MeOH eluted from the column)

4-acetoxy-diamantane-9-ol: 35 mg (2%) via the CH₂Cl₂:ethyl acetate separation (R_{f}=0.24), as well as further purification over silica gel with ethyl acetate as mobile solvent (R_{f}=0.43) and subsequent HPLC purification on a diol phase with t-butyl-methylether: hexane (8:2) as mobile solvent

### Spectroscopic data for 2-chloro-N-[9-(2,2,2)-trifluoroethoxy-diamantane-4-yl]-acetamide

¹H-NMR (400 MHz, CDCl₃) δ: 6.24 (1H, br s, NH), 3.94 (2H, s), 3.79 (2H, q, J=8.8 Hz), 2.10-2.00 (9H, m), 1.91 (3H, br s), 1.81-1.76 (6H, m)

¹³C-NMR (100 MHz, CDCl₃) δ: 164.9, 124.3 (q, J=277.7 Hz), 73.0, 59.3 (q, J=34.2 Hz), 50.8, 42.9, 40.50, 40.46, 38.3, 37.4

¹⁹F-NMR (376 MHz, CDCl₃) δ: −74.55

IR data (KBr): 3294.3, 2931.9, 2893.5, 2862.4, 1690.5, 1663.9, 1555.3, 1444.9, 1411.3, 1353.0, 1291.8, 1172.1, 1109.6, 965.2, 799.3 cm⁻¹

Melting point: 148° C.

HRMS: calculated for C₁₈H₂₃ClF₃NO₂: 377.1369. found: 377.1346

C, H, N: calculated for C₁₈H₂₃ClF₃NO₂ (377.83): C, 57.22; H, 6.14; N, 3.71. found: C, 56.91, H, 6.04, N, 3.40

### Spectroscopic data for 4-acetoxy-9-(2,2,2)-trifluoroethoxy-diamantane

¹H-NMR (400 MHz, CDCl₃) δ: 3.79 (2H, q, J=8.8 Hz), 2.16-2.11 (6H, m), 2.03 (3H, br s), 1.99-1.93 (6H, m), 1.80-1.75 (6H, m)

¹³C-NMR (100 MHz, CDCl₃) δ: 170.4, 124.3 (q, J=277.7 Hz), 78.7, 73.1, 59.3 (q, J=34.2 Hz), 40.4, 40.3, 38.7, 38.4

¹⁹F-NMR (376 MHz, CDCl₃) δ: −74.56

IR data (KBr): 2928.7, 2887.3, 1725.2, 1468.2, 1444.6, 1369.5, 1349.9, 1294.7, 1282.0, 1237.8, 1151.3, 1115.2, 1078.5, 1017.9, 977.5, 948.3, 691.7 cm⁻¹

Melting point: 127° C.

HRMS: calculated for C₁₈H₂₃F₃O₃: 344.1599. found: 344.1571

C, H, N: calculated for C₁₈H₂₃F₃O₃ (344.37): C, 62.78, H, 6.73. found: C, 62.52, H, 6.65

### Spectroscopic data for 2-chloro-N-[9-acetoxy-diamantane-4-yl]-acetamide

¹H-NMR (600 MHz, CDCl₃) δ: 6.24 (1H, br s, NH), 3.94 (2H, s), 2.14-2.11 (6H, m), 2.05-2.03 (6H, m), 2.01 (3H, br s), 1.98-1.95 (6H, m)

¹³C-NMR (150 MHz, CDCl₃) δ: 170.4, 164.9, 78.8, 50.9, 42.9, 40.64, 40.58, 38.6, 37.4, 22.7

IR data (KBr): 3412.8, 2886.1, 1723.1, 1671.4, 1524.5, 1447.8, 1354.6, 1260.7, 1232.8, 1082.7, 1027.1, 859.2, 755.3, 520.4 cm⁻¹

Melting point: 195° C.

HRMS: calculated for C₁₈H₂₄ClNO₃: 337.1445. found: 337.1416

C, H, N: calculated for C₁₈H₂₄ClNO₃ (337.84): C, 63.99; H, 7.16; N, 4.15. found: C, 63.85, H, 7.01, N, 3.75

Spectroscopic data for 4-acetoxy-diamantane-9-ol:

¹H-NMR (400 MHz, CDCl₃) δ: 2.16-2.11 (6H, m), 2.01-1.92 (9H, m), 1.76-1.72 (6H, m), 1.35 (1H, s, OH)

¹³C-NMR (100 MHz, CDCl₃) δ: 170.4, 79.0, 67.1, 44.5, 40.5, 38.7, 38.5, 22.7

IR data (KBr): 3586.9, 3443.5, 2897.7, 2884.6, 1726.4, 1447.7, 1366.6, 1346.8, 1260.4, 1234.0, 1122.0, 1083.1, 1027.8, 958.1 cm⁻¹

Melting point: 151° C.

HRMS: calculated for C₁₆H₂₂O₃: 262.1569. found: 262.1552

C, H, N: calculated for C₁₆H₂₂O₃ (262.34): C, 73.25, H, 8.45. found: C, 73.08, H, 8.37

### Embodiment 4

### Production of 9-(2,2,2-trifluoroethoxy)-diamantane-4-amine

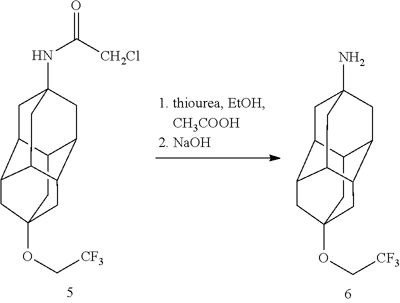

0.48 mmol (180 mg) 2-chloro-N-[9-(2,2,2-trifluoroethoxy)-diamantane-4-yl]-acetamide 5 with a purity of >98% was heated under reflux with 49.5 mg (0.65 mmol) thiourea, 4 ml ethanol and 1.4 ml glacial acetic acid for 16.5 h. Subsequently, the reaction solution was diluted with 25 ml distilled water and made alkaline with 4 ml 10% NaOH. The aqueous solution was extracted three times with, respectively, 20 ml CHCl₃ and the unified organic phases were washed twice with, respectively, 20 ml distilled water, as well as dried over Na₂SO₄. After filtering off the desiccant and removal of the solvent, 133.7 mg (93%) of a weakly yellowish oil was obtained which crystallized after some time.

Spectroscopic data for 9-(2,2,2)-trifluoroethoxy-diamantane-4-amine 6:

¹H-NMR (400 MHz, CDCl₃) δ: 3.80 (2H, q, J=8.8 Hz), 1.93 (3H, br s), 1.86 (3H, br s), 1.80-1.75 (6H, m), 1.65-1.58 (6H, m), 1.16 (2H, NH₂, br s)

¹³C-NMR (100 MHz, CDCl₃) δ: 124.4 (q, J=277.7 Hz), 73.3, 59.3 (q, J=34.2 Hz), 45.7, 45.6, 40.6, 38.4, 38.0

¹⁹F-NMR (376 MHz, CDCl₃) δ: −74.55

IR data (KBr): 3330.2, 2920.7, 2890.6, 1636.9, 1590.8, 1441.6, 1288.6, 1155.9, 1115.0, 1047.8, 1011.7, 976.9, 668.1 cm⁻¹

Melting point: 110-119° C.

HRMS: calculated for C₁₆H₂₃F₃NO: 301.1654. found: 301.1664

### Embodiment 5

### Production of 4-amino-9-diamantane carboxylic acid from 9-(2,2,2-trifluoroethoxy)-diamantane-4-amine

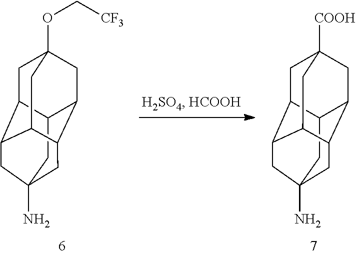

2.3 mmol (700 mg) 9-(2,2,2-trifluoroethoxy)-diamantane-4-amine 6 with a purity of >98% was added to an ice-cooled mixture of 40 ml concentrated sulfuric acid and three ml 100% formic acid. Subsequently, another 7 ml 100% formic acid was added dropwise within 30 min. Four hours after the complete addition and stirring in the ice-bath, the reaction mixture was poured onto 200 g ice. Thereby, a beige, fine precipitate was obtained after 10 min. This was removed by filtration over a Büchner funnel, washed with approx. 50 ml distilled water, and dried over phosphorous pentoxide in a desiccator. Thereby, 453.2 mg of the weakly beige amino acid 7 in the form of the sulfate salt was obtained.

### Spectroscopic data for 4-amino-9-diamantane carbolxylic acid 7

¹H-NMR (400 MHz, NaOD in D₂O) δ: 1.87-1.74 (12H, m), 1.61-1.56 (6H, m)

¹³C-NMR (100 MHz, NaOD in D₂O) δ: 192.7, 49.7, 49.1, 44.6, 44.1, 42.2, 40.7

IR data (KBr): 3449.0, 2890.1, 2695.6, 2639.3, 2584.7, 2183.6, 1696.9, 1532.9, 1512.1, 1469.7, 1446.9, 1384.8, 1292.0, 1251.1, 1137.0, 1082.5, 1036.7, 620.2, 599.7, 464.6 cm⁻¹

Melting point: >310° C.

HRMS: calculated for C₁₅H₂₁NO₂: 247.1572. found: 247.1573 (as zwitterion)

### Embodiment 6

### Production of 4-trifluoroacetoxy-9-(2-chloroacetylamino)-diamantane from 2-chloro-N-[9-(2,2,2-trifluoroethoxy)-diamantane-4-yl]-acetamide

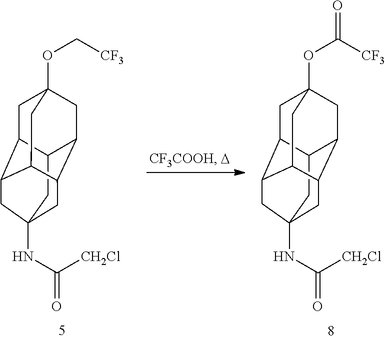

1.06 mml (400 mg) 2-chloro-N-[9-(2,2,2-trifluoroethoxy)-diamantane-4-yl]-acetamide 5 with a purity of >99% was dissolved in 6 ml trifluoroacetic acid and heated under reflux for 4 h. Subsequently, the solvent was removed to dryness using a rotary evaporator. The colorless, solid raw product was separated over silica gel with diethyl ether as mobile solvent. Thereby, 402 mg (97%, R_{f}=0.45) of the pure product was obtained as colorless solid.

### Spectroscopic data for 4-trifluoroacetoxy-9-(2-chloroacetylamino)-diamantane 8

¹H-NMR (400 MHz, CDCl₃) δ: 6.27 (1H, s, NH), 3.96 (2H, s), 2.25-2.20 (6H, m), 2.17-2.06 (9H, m), 2.02 (3H, br s)

¹³C-NMR (100 MHz, CDCl₃) δ: 165.0, 156.0 (q, J=41.3 Hz), 114.3 (q, J=287.8 Hz), 85.4, 50.6, 42.9, 40.3, 40.2, 38.7, 37.1

¹⁹F-NMR (376 MHz, CDCl₃) δ: −75.75

IR data (KBr): 3415, 2941, 2903, 1767, 1674, 1530, 1213, 1164, 1077, 853, 775 cm⁻¹

Melting point: 172° C.

HRMS: calculated for C₁₈H₂₁ClF₃NO₃: 391.1162. found: 391.1163

C, H, N: calculated for C₁₈H₂₁ClF₃NO₃ (391.81): C, 55.18; H, 5.40; N, 3.57. found: C, 55.21, H, 5.27, N, 3.48

### Embodiment 7

### Production of 4-aminodiamantane-9-ol from 4-trifluoroacetoxy-9-(2-chloroacetylamino)-diamantane

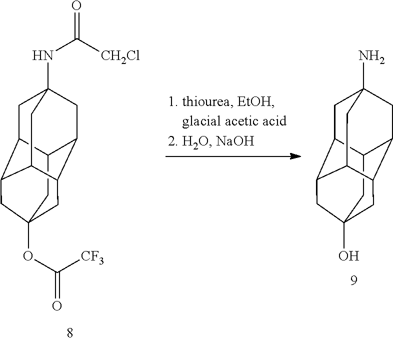

1.62 g (4.12 mmol) 4-trifluoroacetoxy-9-(2-chloroacetylamino)-diamantane 8 was mixed with 474 mg (6.23 mmol) thiourea and dissolved in a mixture of 20 mL ethanol and 15 ml acetic acid. The solution was heated under reflux for 4 hours and diluted with 60 mL of 20% aqueous NaOH solution after cooling to room temperature. After extracting four times with, respectively, 80 mL CHCl₃ and washing twice with, respectively, 80 mL distilled water and drying over anhydrous Na₂SO₄, 781 mg (86%) of the aminoalcohol 9 was obtained in the form of a colorless solid.

### Spectroscopic data for 4-amino-diamantane-9-ol

¹H-NMR (400 MHz, CDCl₃) δ: 1.89-1.79 (6H, m), 1.74-1.68 (6H, m), 1.61-1.56 (6H, m), 1.49-1.24 (3H, m, OH+NH₂)

¹³C-NMR (100 MHz, CDCl₃) δ: 67.2, 45.8, 45.7, 44.8, 38.8, 37.9

IR data (KBr): 3241, 2920, 2887, 2849, 1584, 1470, 1439, 1352, 1254, 1122, 1082, 1045, 1032, 970, 919 cm⁻¹

Melting point: 240-244° C.

HRMS: calculated for C₁₄H₂₁NO: 219.1623. found: 219.1628

C, H, N: calculated for C₁₄H₂₁NO (219.32): C, 76.67; H, 9.65; N, 6.39. found: C, 76.57; H, 9.65; N, 6.33

### Embodiment 8

### Production of 4-nitrodiamantane-9-ol from 4-aminodiamantane-9-ol

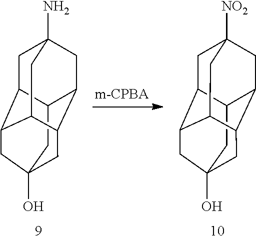

1.74 mmol (300 mg) meta-chloroperbenzoic acid (m-CPBA) was dissolved in 20 mL 1,2-dichloroethane and heated under reflux. For this purpose, a solution of 0.46 mmol (100 mg) 4-amino-diamantane-9-ol 9 was added dropwise to 10 mL 1,2-dichloroethane and rinsed with 5 mL 1,2-dichloroethane. Subsequently, the clear solution was heated under reflux for 3 h. Subsequently, it was cooled to room temperature and the organic phase was washed three times with respectively 60 mL 10% aqueous NaOH solution, as well as dried over Na₂SO₄. After filtering off the desiccant and removal of the solvent, a colorless, solid crude product was obtained. This was purified over silica gel with ether as mobile solvent. Thereby, 101.3 mg (89%, R_{f}=0.19) of the pure nitro alcohol 10 was obtained as colorless solid.

### Spectroscopic data for 4-nitrodiamantane-9-ol

¹H-NMR (400 MHz, CDCl₃) δ: 2.22-2.17 (6H, m), 2.03-1.92 (6H, m), 1.74-1.68 (6 H, m), 1.33 (1H, br s, OH)

¹³C-NMR (100 MHz, CDCl₃) δ: 83.4, 66.7, 44.3, 40.3, 38.2, 37.5

IR data (KBr): 3311.8, 2921.5, 2885.4, 1529.5, 1474.8, 1445.6, 1364.4, 1251.6, 1107.3, 1082.1, 1050.5, 868.8, 802.9 cm⁻¹

Melting point: 172° C.

C, H, N: calculated for C₁₄H₁₉NO₃ (249, 31): C, 67.45; H, 7.68; N, 5.62. found: C, 67.42, H, 7.69; N, 5.52

### Embodiment 9

### Production of 4-amino-9diamantane carbolxylic acid from 4-aminodiamantane-9-ol

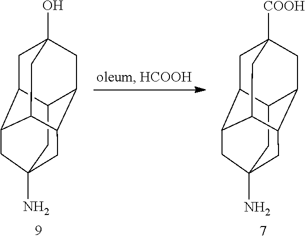

20 mL oleum was reacted under cooling in an ice-salt bath with 1 mL 100% formic acid. Under stirring, 200 mg (0.91 mmol) 4-amino-9-diamantanol, and, subsequently, 1 mL formic acid were added. After 15 min, a clear solution was obtained and during 1 h another 3 mL HCOOH was added dropwise. After stirring at room temperature for one more hour, the solution was poured onto 150 g ice and carefully reacted with 60 mL 30% NaOH solution. Hereby, a colorless precipitate was obtained which was removed by filtration over a Büchner funnel, washed with 30 mL distilled water and dried over P₂O₅. Hereby, 190 mg of the amino acid 7 was obtained as sulfate salt.

Spectroscopic data for 4-amino-9-diamantane carbolxylic acid are listed in embodiment 5.

### Embodiment 10

### Production of 4-amino-9-diamantanecarboxylic acid methyl ester from 4-aminodiamantane-9-ol

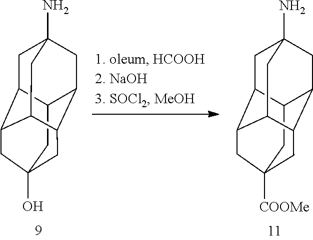

250 mg (1.14 mmol) 4-amino-9-diamantanol 9 was dissolved in 20 mL oleum and cooled in the ice-salt bath at approx. 0° C. Within 1 h, 7 mL 100% formic acid was added dropwise to that solution. Subsequently, it was stirred at room temperature for 1 h and subsequently poured on 100 g ice. 40 mL of 30% NaOH solution was added to the clear solution, so that a colorless precipitate was obtained. The precipitate was removed by filtration over a Büchner funnel, washed with 30 mL distilled water and dried over P₂O₅. The obtained 242 mg of the crude product was subsequently heated under reflux for 30 min with 3 mL thionyl chloride and 15 drops pyridine as catalyst. Subsequently, the excess SOCl₂ was distilled off under vacuum and the crude product reacted with 3 mL absolute methanol (strong gas formation). After stirring for 20 min, the solvent was removed and the oily crude product reacted with 15 mL saturated NaHCO₃ solution and 50 mL distilled water. The aqueous phase was extracted three times with, respectively, 60 ml CHCl₃ and the unified organic phases were washed with 60 ml distilled water, and dried over Na₂SO₄. After filtering off the desiccant and removal of the solvent, 175 mg of a brown solid was obtained. This was sublimated in high vacuum for purification, wherein 112 mg (0.43 mml, 38%) of the pure product was obtained.

### Spectroscopic data for 4-amino-9-diamantanecarboxylic acid methyl ester 11

¹H-NMR (600 MHz, CDCl₃) δ: 3.68 (3H, s), 1.94-1.89 (6H, m), 1.87 (3H, br s), 1.81 (3H, br s), 1.63-1.58 (6H, m), 1.24 (2H, NH₂, br s)

¹³C-NMR (150 MHz, CDCl₃) δ: 178.2, 51.6, 46.1, 45.8, 38.86, 38.84, 38.1, 36.1

IR data (KBr): 3344.3, 3280.7, 2956.4, 2915.9, 2879.8, 2864.0, 1730.0, 1596.5, 1461.5, 1442.2, 1353.7, 1280.3, 1228.5, 1095.3, 1043.7, 867.1 cm⁻¹

Melting point: 117° C.

HRMS: calculated for C₁₆H₂₃NO₂: 261.1729. found: 261.1731

C, H, N: calculated for C₁₆H₂₃NO₂ (261.36): C, 73.53; H, 8.87; N, 5.36. found: C, 73.41, H, 8.94, N, 5.03

### Embodiment 11

### Production of 4-amino-9-diamantane carbolxylic acid-hydrochloride from 4-amino-9-diamantanecarboxylic acid methyl ester

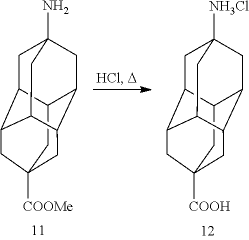

0.23 mmol (60 mg) 4-amino-9-diamantanecarboxylic acid methyl ester 11 was dissolved in 3 mL concentrated hydrochloric acid and heated under reflux for 3 h. Subsequently, it was cooled to room temperature, wherein colorless crystals precipitated. These were drawn off and dried using a rotary evaporator. Hereby, 34.2 mg of the colorless product was obtained. By concentrating the filtrate to dryness, another 25.2 mg of the product was obtained. In total, 59.4 mg (91%) 4-amino-9-diamantane carbolxylic acid hydrochloride 12 was obtained.

### Spectroscopic data for 4-amino-9-diamantane carbolxylic acid hydrochloride

¹H-NMR (400 MHz, DMSOD₆) δ: 12.09 (1H, s, COOH), 8.06 (3H, s, NH₃), 1.86 (3 H, br s), 1.81-1.71 (15H, m)

¹³C-NMR (100 MHz, CDCl₃) δ: 178.5, 49.9, 40.1 (aus DEPT135), 38.4, 37.6, 36.6, 35.3

IR data (KBr): 3411.8, 3147.4, 2925.5, 2890.3, 2860.8, 2612.7, 2552.1, 2459.2, 1978.6, 1715.6, 1699.1, 1614.5, 1598.4, 1517.5, 1352.7, 1288.1, 1236.3, 1200.7, 1096.2 cm⁻¹

Melting point: >350° C.

C, H, N: calculated for C₁₅H₂₂ClNO₂ (283, 79): C, 63.48; H, 7.81; N, 4.94. found: C, 63.30; H, 8.02; N, 4.75

### Embodiment 12

### Production of 4-(2,2,2-trifluoro-1-trifluoromethyl-ethoxy)-diamantane-9-ol

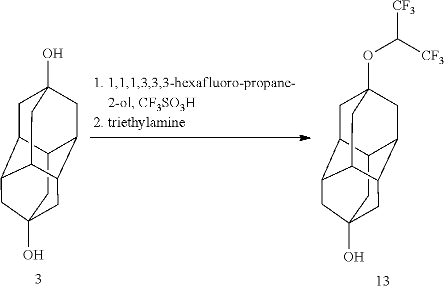

1.13 mmol (250 mg) 4,9-diamantanediol 3 with a purity of >99% was added to 8 mL 1,1,1,3,3,3-hexafluoropropane-2-ol. 3 μL Trifluoromethanesulfonic acide was added to that. The colorless suspension was stirred for 45 min at room temperature and subsequently reacted with 50 μL triethylamine. After removal of the solvent, the solid, colorless crude product was separated over silica gel with ethyl acetate:hexane (8:2) as mobile solvent. Thereby, 134 mg (32%, R_{f}=0.38) of the pure monoether 13 was obtained as colorless solid. Furthermore, 101.7 mg (17%, R_{f}=0.77) of the pure diether and 107.9 mg (43%, R_{f}=0.18) of the starting material were obtained.

### Spectroscopic data for 4-(2,2,2-trifluoro-1-trifluoromethyl-ethoxy)-diamantane-9-ol 13

¹H-NMR (600 MHz, CDCl₃) δ: 4.34 (1H, septet, J=6.0 Hz), 1.97 (3H, br s), 1.92 (3H, br s), 1.82-1.78 (6H, m), 1.75-1.71 (6H, m), 1.33 (1H, s, OH)

¹³C-NMR (150 MHz, CDCl₃) δ: 121.6 (q, J=283.7 Hz), 77.5, 68.2 (septet, J=32.2 Hz), 66.9, 44.3, 40.9, 38.54, 38.52

¹⁹F-NMR (376 MHz, CDCl₃) δ: −73.34

IR data (KBr): 3278.9, 2924.5, 2880.6, 2862.2, 1445.6, 1359.6, 1282.3, 1251.3, 1194.9, 1113.9, 1096.4, 1048.4, 1005.2, 957.7, 890.1, 685.3 cm⁻¹

Melting point: 163° C.

HRMS: calculated for C₁₇H₂₀F₆O₂: 370.1368. found: 370.1343

C, H, N: calculated for C₁₇H₂₀F₆O₂ (370, 33): C, 55.14, H, 5.44. found: C, 55.12, H, 5.31

### Embodiment 13

### Production of 1-(2,2,2-triflluoroethoxy)-diamantane-6-ol

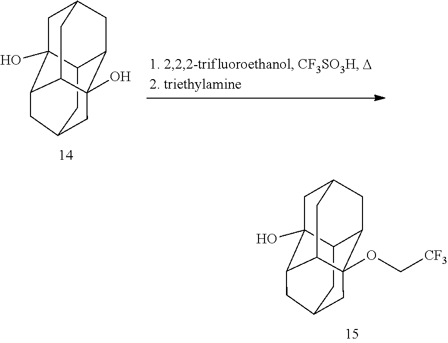

1.13 mmol (250 mg) 1,6-diamantanediol 14 with a purity of >99% was dissolved in 40 ml 2,2,2-trifluoroethanol and reacted with 25 μL CF₃SO₃H at 40° C. in a water-bath. After stirring for 20 min at the same temperature the acid was neutralized with 100 μL triethylamine and the solvent removed to dryness. The colorless, solid raw product was separated over silica gel with ethyl acetate:hexane 8:2 as mobile solvent. Thereby, 220 mg (64%, R_{f}=0.54) of the pure monoether 15 was obtained as colorless solid. Furthermore, 64 mg (15%, R_{f}=0.72) of the pure diether was obtained.

Spectroscopic data for 1-(2,2,2)-trifluoroethoxy-diamantane-6-ol 15:

¹H-NMR (400 MHz, CDCl₃) δ: 3.65 (2H, q, J=8.4 Hz), 2.14-1.97 (6H, m), 1.89 (2H, s), 1.75 (2H, s), 1.63-1.57 (4H, m), 1.36-1.27 (4H, m), 1.21 (1H, OH, br s)

¹³C-NMR (100 MHz, CDCl₃) δ: 124.5 (q, J=277.7 Hz), 75.6, 70.3, 58.8 (q, J=34.2 Hz), 46.1, 44.8, 41.8, 39.6, 31.5, 31.4, 29.4, 29.1

¹⁹F-NMR (376 MHz, CDCl₃) δ: −74.14

IR data (KBr): 3264.2, 2913.9, 2903.9, 1463.6, 1346.3, 1290.8, 1271.8, 1157.3, 1105.5, 1007.6, 966.7, 895.3, 679.2 cm⁻¹

Melting point: 124° C.

HRMS: calculated for C₁₆H₂₁F₃O₂: 302.1494. found 302.1475

C, H, N: calculated for C₁₆H₂₁F₃O₂ (302, 32): C, 63.56, H, 7.00. found: C, 63.64, H, 7.00

### Embodiment 14

### Production of 9-(2,2,2-trifluoroethoxy)-triamantane-15-ol

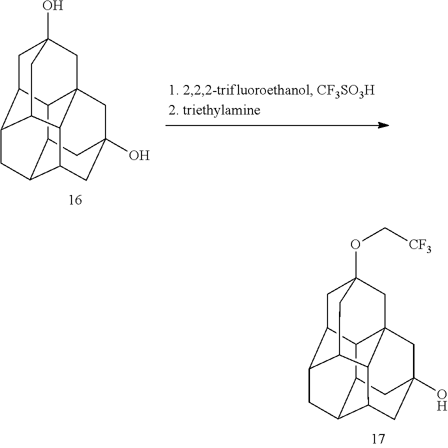

0.15 mmol (40.8 mg) 9,15-anti-tetramantanediol 16 with a purity of >99% was dissolved in 20 ml 2,2,2-trifluoroethanol and reacted with 5 μL CF₃SO₃H at room temperature. After stirring for 14 h at the same temperature, the acid was neutralized with 10 μL triethylamine and the solvent removed to dryness. The oily raw product was separated over silica gel with ethyl acetate:hexane 8:2 as mobile solvent. Thereby, 28.0 mg (53%, R_{f}=0.49) of the pure monoether 17 was obtained as colorless solid. Furthermore, 17.5 mg (27%, R_{f}=0.72) of the pure diether and 5.1 mg (13%, R_{f}=0.15) of the starting material were obtained.

### Spectroscopic data for 9-(2,2,2)-trifluoroethoxy-diamantane-15-ol 17

¹H-NMR (400 MHz, CDCl₃) δ: 3.79 (2H, q, J=8.8 Hz), 2.05-1.92 (4H, m), 1.81-1.60 (12H, m), 1.44-1.31 (7H, m)

¹³C-NMR (100 MHz, CDCl₃) δ: 124.4 (q, J=277.7 Hz), 73.9, 67.9, 59.3 (q, J=34.2 Hz), 52.0, 47.7, 45.2, 44.5, 41.1, 39.5, 39.2, 38.5, 36.3, 33.8, 33.5

¹⁹F-NMR (376 MHz, CDCl₃) δ: −74.54

IR data (KBr): 3304.4, 2917.7, 2900.3, 2874.7, 2850.7, 1443.9, 1339.3, 1281.4, 1156.7, 1146.1, 1122.1, 1112.0 cm⁻¹

Melting point: 153° C.

HRMS: calculated for C₂₀H₂₅F₃O₂: 354.1807. found: 354.1773

C, H, N: calculated for C₂₀H₂₅F₃O₂ (354.41): C, 67.78, H, 7.11. found: C, 67.69, H, 7.16

### Embodiment 15

### Production of 6-(2,2,2-trifluoroethoxy)-anti-tetramantane-13-ol

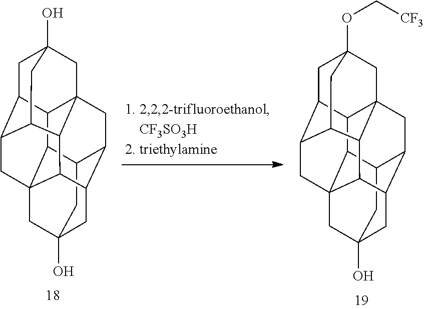

0.3 mmol (97.3 mg) 6,13-anti-tetramantanediol 18 with a purity of >99% was dissolved in 36 ml 2,2,2-trifluoroethanol and reacted with 9 μL CF₃SO₃H at room temperature. After stirring for 2 h at the same temperature, the acid was neutralized with 20 μL triethylamine and the solvent removed to dryness. The colorless, solid raw product was separated over silica gel with ethyl acetate:hexane 8:2 as mobile solvent. Thereby, 75.7 mg (62%, R_{f}=0.48) of the pure monoether 19 was obtained as colorless solid. Furthermore, 34.2 mg (23%, R_{f}=0.77) of the pure diether and 12.2 mg (13%, R_{f}=0.32) of the starting material were obtained.

Spectroscopic data for 6-(2,2,2)-trifluoroethoxy-anti-tetramantane-13-ol 19:

¹H-NMR (400 MHz, CDCl₃) δ: 3.79 (2H, q, J=8.8 Hz), 1.97-1.87 (4H, m), 1.80-1.63 (10H, m), 1.45-1.27 (13H, m)

¹³C-NMR (150 MHz, CDCl₃) δ: 124.4 (q, J=277.7 Hz), 74.3, 68.3, 59.2 (q, J=34.7 Hz), 51.3, 47.1, 45.4, 45.2, 45.1, 44.3, 44.1, 40.9, 40.3, 39.9, 35.2, 35.0, 33.6, 33.5

¹⁹F-NMR (376 MHz, CDCl₃) δ: −74.54

IR data (KBr): 3268.0, 2901.5, 2879.5, 1465.4, 1446.5, 1418.8, 1338.8, 1282.6, 1159.0, 1147.9, 1122.7, 1112.6, 1071.1, 1029.0, 1004.3, 991.7, 969.2, 866.3, 763.7 cm⁻¹

Melting point: 169° C.

HRMS: calculated for C₂₄H₂₉F₃O₂: 406.2120. found: 406.2106

1. Functionalized diols of diamondoids according to formula (I), in which one of the two hydroxy groups is masked by a protective group 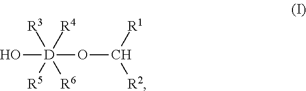
wherein
D represents a diamondoid,
R¹ stands for a linear or branched alkyl group —CnHₚX_{q},
wherein n is a natural number from 1 to 25,
p is an integer between 0 and 50 and q is a natural number between 1 and 51,
and the sum of q and p is (2n+1),
R² stands for hydrogen or a linear or branched alkyl group —CₘHᵣXs,
wherein m is a natural number from 1 to 25,
r and s are integers between 0 and 51,
and the sum of r and s is (2 m+1),
X stands for a halogen selected from fluorine, chlorine or bromine,
R³, R⁴, R⁵ and R⁶ represent, independently of one another:
hydrogen,
a linear or branched alkyl group having 1 to 20 carbon atoms,
a cyclic alkyl group having 3 to 20 carbon atoms,
an aryl group having 6 to 18 carbon atoms,
a heteroaryl group having 5 to 18 ring atoms, of which 1 or 2 atoms are heteroatoms selected from nitrogen, oxygen, sulfur, and the remaining ring atoms are carbon atoms.

2. Functionalized diols of diamondoids according to claim 1, wherein the halogen X in R¹ and R² stands for fluorine.

3. Functionalized diols of diamondoids according to one of claims 1 to 2, wherein R³ and R⁴ stand for hydrogen and R⁵ and R⁶ for a linear or branched alkyl group having 1 to 20 carbon atoms.

4. Functionalized diols of diamondoids according to claims 1 to 3, wherein R³ and R⁴ stand for hydrogen and R⁵ and R⁶ are identical.

5. Functionalized diols of diamondoids according to one of claims 1 to 4, wherein R³ and R⁴ stand for hydrogen, R⁵ and R⁶ are identical and R⁵ and R⁶ stand for a linear or branched alkyl group having 1 to 20 carbon atoms.

6. Functionalized diols of diamondoids according to one of claims 1 to 5, wherein the protective group according to formula (II) 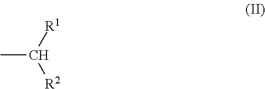
comprises a total of 2 to 12 carbon atoms.

7. Functionalized diols of diamondoids according to one of claims 1 to 6, wherein the protective group —CHR¹R² is selected from —O—CH₂—CF₃, —O—CH₂—CF₂H, —O—CH₂—CF₂—CF₃, —O—CH₂—CF₂—CF₂H, —O—CH—(CF₃)₂, —O—CH₂—(CF₂)₂—CF₃, —O—CH₂—(CF₂)₂—CF₂H, —O—CH₂—(CF₂)₃—CF₃, —O—CH₂—(CF₂)₃—CF₂H.

8. Functionalized diols of diamondoids according to one of claims 1 to 7, wherein D is a lower diamondoid selected from adamantane, diamantane and triamantane.

9. Method for producing functionalized diols of diamondoids comprising the following steps:
a) mixing a diamondoid diol DR³R⁴R⁵R⁶(OH)₂ with a halogenated alcohol CHOHR¹R², wherein D, R¹, R², R³, R⁴, R⁵ and R⁶ are as defined above,
b) adding a catalyst acid,
c) stirring at −20° C. to 100° C.,
d) ending the reaction by adding an amine or water,
e) isolating the crude end product,
f) purifying the end product.

10. Method for the production of functionalized diols of diamondoids according to claim 9, wherein the alcohol CHOHR¹R² is selected from —O—CH₂—CF₃, —O—CH₂—CF₂H, —O—CH₂—CF₂—CF₃, —O—CH₂—CF₂—CF₂H, —O—CH—(CF₃)₂, —O—CH₂—(CF₂)₂—CF₃, —O—CH₂—(CF₂)₂—CF₂H.

11. Method for the production of functionalized diols of diamondoids according to one of claims 9 and 10, wherein the catalyst acid in step c) is selected from CF₃SO₃H and p-toluenesulfonic acid.

12. Method for the production of functionalized diols of diamondoids according to one of claims 9 to 11, wherein the ending of the reaction according to step d) takes place by adding an amine.

13. Method for the production of functionalized diols of diamondoids according to one of claims 9 to 11, wherein the ending of the reaction according to step d) takes place by adding water.

14. Use of functionalized diols of diamondoids according to one of the claims 1 to 8 for the production of monoether amides according to formula (V) 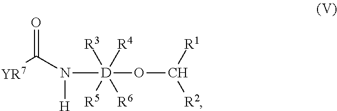
wherein R⁷ stands for a linear, branched or cyclic alkylene group —CₜH₂ₜ—, t is a natural number from 1 to 11, Y is a halogen atom selected from fluorine, chlorine, bromine, iodine, and R¹ to R⁶ are as defined above, wherein a functionalized diol of a diamondoid is reacted with a nitrile YR⁷—CN in a Ritter reaction in the presence of concentrated sulfuric acid to the monoether amide.

15. Use of monoether amides according to claim 14, wherein R² stands for a hydrogen atom, in a method for the production of aminoalcohols of diamondoids, comprising the steps:
a) reaction of the monoether amide with trifluoroacetic acid,
b) heating with thiourea, ethanol and glacial acetic acid,
c) making the reaction solution alkaline with an aqueous alkaline solution,
d) isolation and purification of the aminoalcohol obtained in this way.

16. Use of aminoalcohols according to claim 15 in a method for the production of aminocarboxylic acids of diamondoids, comprising the steps:
a) Reaction of the aminoalcohol with oleum and concentrated formic acid at temperatures between −20° C. and 0° C.,
b) precipitation of the crude aminocarboxylic acid salt with an alkaline solution,
c) isolation, purification and drying of the aminocarboxylic acid salt,
d) reaction with thionyl chloride and methanol to aminocarboxylic acid methyl ester,
e) cleavage of the ester by heating with a strong mineral acid,
f) cleavage, purification and drying of the aminocarboxylic acid.

17. Use of monoether amides according to claim 14 in a method for the production of aminocarboxylic acids of diamondoids, comprising the steps:
a) heating the monoethers amide with thiourea, ethanol and glacial acetic acid,
b) dilution of the reaction solution with water,
c) making the reaction solution alkaline with an alkaline solution,
d) isolation and purification of the monoether amine obtained in this way,
e) reaction of the monoether with concentrated sulfuric acid and 100% formic acid at temperatures between −20° C. and 0° C.,
f) pouring reaction mixture from step e) onto ice, isolation and purification of the aminocarboxylic acid obtained in this way.
